(19) United States
(12) Patent Application Publication
(10) Pub. No.: US 2024/0277364 A1
(43) Pub. Date: Aug. 22, 2024

(54) PULSATILE ASPIRATION SYSTEM USING A STATIC VACUUM SOURCE WITH INTERMITTENT INJECTION OF A PRESSURE PULSE

(71) Applicant: Neuravi Limited, Galway (IE)

(72) Inventor: Brendan CASEY, Galway (IE)

(73) Assignee: Neuravi Limited, Galway (IE)

(21) Appl. No.: 18/441,798

(22) Filed: Feb. 14, 2024

Related U.S. Application Data
(60) Provisional application No. 63/447,506, filed on Feb. 22, 2023.

(51) Int. Cl. A61B 17/221; A61B 17/00; A61M 25/00

(52) U.S. Cl. CPC A61B 17/221; A61B 2017/00022; A61B 2017/00154; A61B 2017/2215; A61M 2025/0002; A61M 2025/0004; A61M 25/003; A61M 2025/0031; A61M 2025/0039; A61M 25/0068; A61M 2205/3327; A61M 2205/3351

(57) ABSTRACT

A pulsatile aspiration system is herein disclosed that includes a catheter and pump system. The catheter includes an outer wall, a proximal section, a distal section, and an internal wall dividing the proximal section into a central main lumen and an auxiliary lumen. The pump system is in fluid communication with the catheter and independently controls the negative fluid pressure and the positive fluid pressure in the central main lumen and the auxiliary lumen so as to produce at the distal end of the catheter a pulsatile aspiration waveform variable between a maximum aspiration pressure and a maximum positive pressure. The maximum aspiration pressure is producible at the distal end of the catheter when the resulting pressure is exclusively negative fluid pressure, and the maximum positive pressure is producible at the distal end of the catheter when the positive fluid pressure counterbalances that of the negative fluid pressure.

### CROSS-REFERENCE TO RELATED APPLICATION

This application claims the benefit of U.S. Provisional Patent Application No. 63/447,506 filed Feb. 22, 2023, the disclosure of which is incorporated herein by reference in its entirety.

### FIELD

The present disclosure generally relates to systems and methods used during thrombectomy procedures for the capture and removal of occlusions or clots. Specifically, the present disclosure relates to a pulsatile aspiration system for the capture and removal of occlusions or clots in a vessel where the pulsatile aspiration pressure waveform includes static vacuum pressure produced using a static vacuum pressure source and injection of a pressure pulse by a positive pressure source.

### BACKGROUND

Clots are essentially living polymers, comprising a matrix of intertwined and cross-linked fibrin strands within which are situated red and white blood cells, platelets and numerous other proteins and components. The mechanical properties of a clot are strongly influenced by the relative percentages of fibrin and red blood cells, and that clots with a high (and highly organized) fibrin content and low red blood cell content tend to be much firmer and more cohesive than clots of a higher red cell content. Such clots have also been found to have a higher coefficient of friction, or in other words to be “stickier”. These firm and sticky clots can be very challenging to remove from a vessel.

Clots with a low fibrin content and high red cell content have been found to be less cohesive and more friable and to have a lower coefficient of friction than the more organized fibrin rich clots previously described. These properties mean that such clots may be easier to dislodge from the site of occlusion, but may tend to fragment during the retrieval process, with the consequent risk of loss of clot fragments into distal or new vascular territories.

Attempts to aspirate such clots into a catheter can be very challenging as the clot must be deformed in order to fit into the catheter lumen, and the energy required to deform such clots is not easily attained by aspiration. The high frictional coefficient of these clot types adds further to the challenge of aspirating them into the distal mouth of a catheter. Even maintaining a suction grip on such clots so that they can be retracted to the safety of a more proximal guide or sheath is very difficult, as these firm clots do not tend to deform and reshape easily and thus do not readily conform to the shape of the catheter tip in order to effect a seal and consequent suction grip.

Rather than static aspiration, which applies a constant vacuum pressure to the distal tip or end of the aspiration catheter, pulsatile aspiration applies a static vacuum pressure and a pressure wave having a pressure higher than that of the static vacuum pressure. During cycles under the static vacuum pressure the clot is drawn in the proximal direction and captured in the distal tip of the aspiration catheter, whereas during cycles of the pressure wave the pressure at the distal tip increases.

One challenge associated with the use of pulsatile aspiration in the capture and removal of the clot is the dampening effect on the waveform along the length of the catheter (due to the catheter's length and inner diameter). While ideally the vacuum waveform varies from, e.g., ambient pressure to full vacuum pressure at a high frequency, this is not possible in conventional systems due to the dampening effect. One option is to generate the pressure waveform at the distal tip of the catheter, but that is difficult to do, particularly while simultaneously maintaining the flexibility and other performance requirements needed from the catheter.

It is therefore desirable to develop an improved pulsatile aspiration system having as few active components as possible that reduce the aforementioned dampening effect and enable the cycling frequency to be maximized.

### SUMMARY

There is provided, in accordance with the disclosed technology, a pulsatile aspiration system comprising a catheter and a pump system. The catheter comprises an outer wall, a proximal section, a distal section, and an internal wall. The outer wall extends in a longitudinal direction from a proximal end to an opposite distal end and defines a passageway therethrough. The proximal section has an associated proximal section of the passageway. The distal section disposed distally relative to the proximal section and has an associated distal section of the passageway. The internal wall extends in the longitudinal direction through the passageway and divides the proximal section of the passageway into (i) a central main lumen defined radially inward relative to the internal wall and (ii) an auxiliary lumen at least partially defined by the internal wall and internal to the outer wall. The central main lumen, through which a first fluid is receivable, is configured to produce one of negative fluid pressure or positive fluid pressure therein. The auxiliary lumen, through which a second fluid is receivable, is configured to produce the other of the positive fluid pressure or the negative fluid pressure therein. A distal portion of the central main lumen and a distal portion of the auxiliary lumen are in fluid communication with one another where the negative fluid pressure and the positive fluid pressure combine as a resulting pressure in the distal section of the passageway. The pump system is in fluid communication with the catheter and independently controls the negative fluid pressure and the positive fluid pressure in the central main lumen and the auxiliary lumen so as to produce at the distal end of the catheter a pulsatile aspiration waveform variable between a maximum aspiration pressure and a maximum positive pressure. The maximum aspiration pressure is producible at the distal end of the catheter when the resulting pressure is exclusively negative fluid pressure, and the maximum positive pressure is producible at the distal end of the catheter when the positive fluid pressure counterbalances that of the negative fluid pressure.

There is further provided, in accordance with the disclosed technology, a method for operating a pulsatile aspiration system including a catheter. The catheter has an outer wall extending in a longitudinal direction from a proximal end to an opposite distal end. The outer wall defines a passageway therethrough. The catheter includes a proximal section with an associated proximal section of the passageway and a distal section disposed distally thereof with an associated distal section of the passageway. The proximal section of the passageway is divided by an internal wall extending in the longitudinal direction therethrough. It is divided into (i) a central main lumen defined radially inward relative to the internal wall and (ii) an auxiliary at least partially defined by the internal wall. The central main lumen, through which a first fluid is receivable, is configured to produce one of negative fluid pressure or positive fluid pressure therein. The auxiliary lumen, through which a second fluid is receivable, is configured to produce the other of the negative fluid pressure or the positive fluid pressure therein. A distal end of the central main lumen and a distal end of the auxiliary lumen are in fluid communication with one another where the negative fluid pressure and the positive fluid pressure combine as a resulting pressure in the distal section of the passageway. The pulsatile aspiration system further includes a pump system in fluid communication with the catheter. The method comprises the step of independently controlling the negative fluid pressure and the positive fluid pressure in the central main lumen and the auxiliary lumen so as to produce at the distal end of the catheter a pulsatile aspiration waveform variable between maximum aspiration pressure and a maximum positive pressure. The maximum aspiration pressure is producible at the distal end of the catheter when the resulting pressure is exclusively negative fluid pressure, while the maximum positive pressure is producible at the distal end of the catheter when the positive fluid pressure counterbalances that of the negative fluid pressure.

### BRIEF DESCRIPTION OF THE DRAWINGS

The above and further aspects of the presently disclosed technology are further discussed with reference to the following description in conjunction with the accompanying drawings, in which like numerals indicate like structural elements and features in various figures. The drawings are not necessarily to scale, emphasis instead being placed upon illustrating principles of the disclosed technology. The figures depict one or more implementations of the inventive devices, by way of example only, not by way of limitation.

FIG. 1A diagrammatically depicts a perspective view of an example of a distal tip of a catheter of a pulsatile aspiration system, in accordance with the disclosed technology;

FIG. 1B diagrammatically depicts a cross-sectional view of the distal tip of FIG. 1A, taken along line I in FIG. 1A, in accordance with the disclosed technology;

FIG. 1C diagrammatically depicts another cross-sectional view of the distal tip of FIG. 1A, taken along line II in FIG. 1A, in accordance with the disclosed technology;

FIG. 2A diagrammatically depicts a perspective view of another example of a distal tip of the catheter of the pulsatile aspiration system, in accordance with the disclosed technology;

FIG. 2B diagrammatically depicts a cross-sectional view of the distal tip of FIG. 2A, taken along line III in FIG. 2A, in accordance with the disclosed technology;

FIG. 2C diagrammatically depicts another cross-sectional view of the distal tip of FIG. 2A, taken along line IV in FIG. 2A, in accordance with the disclosed technology;

FIG. 3 diagrammatically depicts an example of the pulsatile aspiration system, shown with the distal tip of FIG. 1A, in accordance with the disclosed technology;

FIG. 4 diagrammatically depicts a graph of an exemplary pressure waveform at the distal tip of the catheter, in accordance with the disclosed technology; and

FIGS. 5A-5B depict a pulsatile aspiration method, in accordance with the disclosed technology.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

The present disclosure is directed to an improved pulsatile aspiration system and method for use having a reduced dampening effect which allows for increased cycling frequency of aspiration (vacuuming) and a maximum positive pressure. The system described herein provides a highly flexible catheter with high frequency variable pulsatile aspiration.

It is noted that, as used herein, “aspiration pressure” is describes a vacuum or negative pressure. Accordingly, a “maximum aspiration pressure” likewise is synonymous with a “maximum vacuum force”.

The catheter includes two lumens that are connected to a programmable pump system. One lumen is used for vacuum aspiration and the other lumen is used for ambient or positive pressure fluid flow. By controlling the pressure in each lumen, a greater amplitude and frequency of pulsatile waveforms at the distal tip can be achieved than by merely connecting the proximal end of the catheter to the pump.

In accordance with the disclosed technology, the two lumen configuration results in a dual lumen in a proximal and mid-section of the catheter, with a single lumen at the distal tip of the catheter.

In accordance with the disclosed technology, control sensors are integrated therewith, and a stentriever can be used, if needed. For example, a pressure sensor at the distal tip can be used to monitor the pressure and provide a feedback loop to the pump/control system to ensure optimal pulsatile parameters are achieved. Moreover, the pressure sensor also ensures that a net positive pressure is not achieved at the distal tip, as that could push the clot more distal in the vessel (rather than drawing it towards the distal tip).

In use, a vacuum pressure is applied to a first one of the two lumens. In addition to the novel techniques described herein, this lumen can be used in conjunction with standard aspiration techniques. To apply a pulsatile vacuum to the distal tip, the vacuum pressure can be varied in this lumen and a positive fluid pulse can be applied through the second one of the two lumens. The positive pressure combines with the negative pressure from the first lumen to result in a net maximum air pressure. In some examples, this is approximately ambient atmospheric pressure.

By using two lumens, a rapid change in pressure at the distal tip can be achieved by injection of the positive fluid pulse in the second lumen, as the negative pressure in the first lumen does not need to change significantly or at all. Therefore, to go from full vacuuming to the net maximum (e.g., ambient) pressure, only a small volume of applied/injected fluid is required to be injected into the single lumen at the distal tip of the catheter. The control system can calculate the volume of the injected positive fluid pulse based on the applied vacuum pressure level and the pressure reading at the distal tip, ensuring the distal tip pressure does not exceed the net maximum pressure.

Other features of the presently disclosed technology include, but are not limited to, the following.

A clot sensor can be provided in a funnel tip to identify mechanical properties of the clot and determine the waveform parameters applied by the pump. These parameters can change along the length of the clot if the mechanical properties (e.g., fibrin content, red blood cell (RBC) content, or clot impedance) changes.

In certain catheter examples, an inner catheter and an outer catheter can be provided that are advanced and/or retracted independently of one another. By way of example, and as discussed in greater detail below, the outer catheter can first be advanced to the clot face and then the inner catheter forwarded into position without impacting the ability to track the outer catheter. In this example, the inner catheter's diameter can be optimized for use with a microcatheter and/or stentriever.

Additionally, one of the lumens can be positioned such that there is a reception space for the clot to be ingested. The pressure sensor can identify when the clot is fully ingested, e.g., by detecting a change in the pressure waveform, which serves as an indicator to the physician that the catheter can be removed.

Based on the principles described above, certain illustrative examples will now be described of pulsatile aspiration systems in accordance with the presently disclosed technology.

Different exemplary configurations of the pulsatile aspiration system are described in the present disclosure all of which include an aspiration catheter connected via a proximal hub. A static vacuum source, such as a vacuum pump, is in fluid communication with the proximal hub either directly or via inlet tubing. In addition, the pulsatile aspiration system further includes a positive pressure source producing a positive pressure pulse.

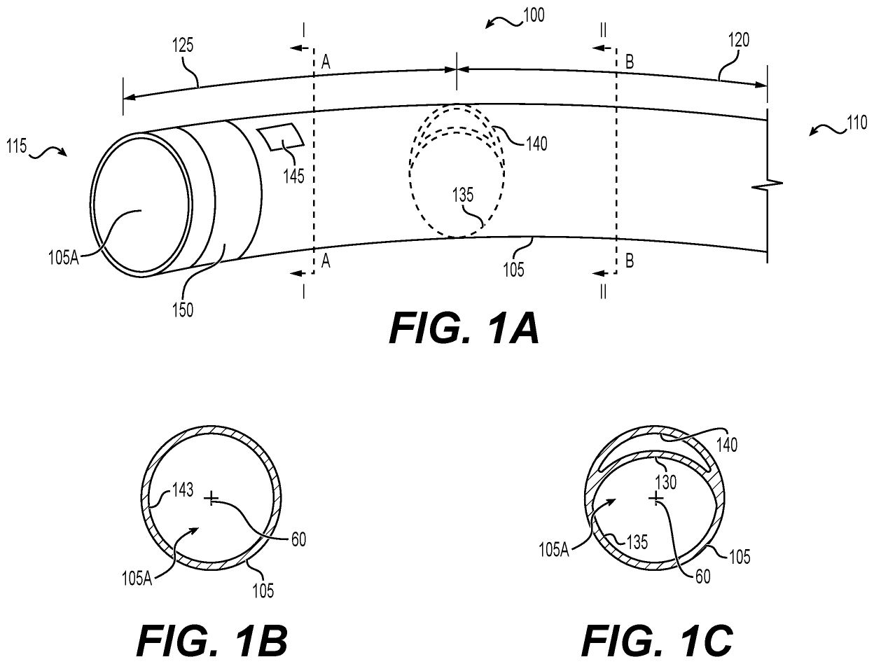

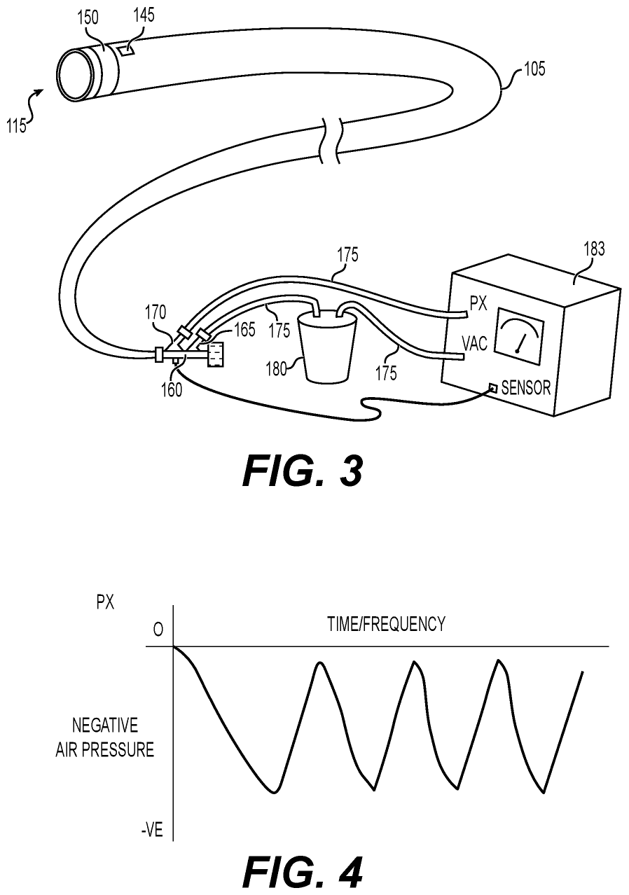

FIG. 1A depicts a perspective view of an example of a distal tip of a catheter of a pulsatile aspiration system. FIG. 1B depicts a cross-sectional view of the distal tip of FIG. 1A, taken along line I in FIG. 1A. FIG. 1C depicts another cross-sectional view of the distal tip of FIG. 1A, taken along line II in FIG. 1A. FIG. 3 diagrammatically depicts an example of the pulsatile aspiration system, shown with the distal tip of FIG. 1A.

With reference now to FIGS. 1A-1C and 3, a distal tip of a catheter 105 is shown, which forms a portion of a pulsatile aspiration system 100 (FIG. 3). The catheter 105 has an outer wall that extends in a longitudinal direction along a longitudinal axis 60 from a proximal end 110 to an opposite free distal end 115, with a passageway 105A being defined therethrough. Referring to FIG. 1A in conjunction with FIG. 3, the proximal end 110 is connected to a hub 160, which is described in greater detail below. The catheter 105 further includes a proximal section 120 that is associated with a proximal section 135, 140 of the passageway 105A and a distal section 125 disposed distally thereof that is associated with a distal section 143 of the passageway 105A and terminates at the distal end 115. In some examples, the distal section 125 can be varied in length between approximately 0 centimeters (where distal section 125 is essentially an extension of the proximal section 120 and extends approximately to the free distal end 115, but optimally at least a few millimeters therefrom) and 5 centimeters. In some examples, the distal section 125 can include a radiopaque marker 150 for tracking a location of the distal tip in the patient's body.

As particularly seen in FIG. 1C, the proximal section 135, 140 of the passageway 105A includes an internal wall 130 that extends proximally in the longitudinal direction through the passageway 105A. The internal wall 130 divides the proximal section 135, 140 of the passageway 105A into a central main lumen 135 and an auxiliary lumen 140, through both of which fluid is receivable. The central main lumen 135 is at least partially defined by the internal wall 130 such that it is defined on one side of the internal wall 130 (e.g., radially inward towards the longitudinal axis 60 relative to the internal wall 130) and is sized so as to be capable of receiving a stentriever. The auxiliary lumen 140 is at least partially defined by the internal wall 130 such that it is arranged on another side of the internal wall 130 (e.g., radially outwards from the longitudinal axis 60 relative to the internal wall 130). The internal wall 130 is designed such that, in cross-section, the areas of central main lumen 135 and auxiliary lumen 140 have a predetermined ratio relative to one another to maximize space for clot ingestion. In some examples, the ratio of central main lumen area to auxiliary lumen area is at least 70:30, such as 90:10 or 95:5. In contrast, and as particularly seen in FIG. 1B, the distal section 143 of the passageway 105A is undivided. However, as allude to above, it will be appreciated that, in some examples, the distal section 125/143 (as shown in the figures) can be configured essentially the same as the proximal section 120 by extending the internal wall 130 all the way to the free distal end 115.

In this example, the auxiliary lumen 140 is arranged eccentrically of the central main lumen 135. Moreover, the internal wall 130 is permanently fixed in position within and dividing the proximal section of the passageway 105A of the catheter 105 into the central main lumen 135 and the auxiliary lumen 140 on opposite sides of the internal wall 130 in a radial direction.

In some examples, in the proximal section 120, the central main lumen 135 produces negative fluid pressure therein by way of a fluid (e.g., blood) and pump system that is described in greater detail below. In these examples, the auxiliary lumen 140 produces positive fluid pressure therein by way of a biocompatible fluid and pump system that is described in greater detail below.

It will be appreciated that, in other examples and in accordance with the presently disclosed technology, the positive and negative pressures can be reversed such that negative fluid pressure is produced in the auxiliary lumen 140 and positive fluid pressure is produced in the main lumen 135.

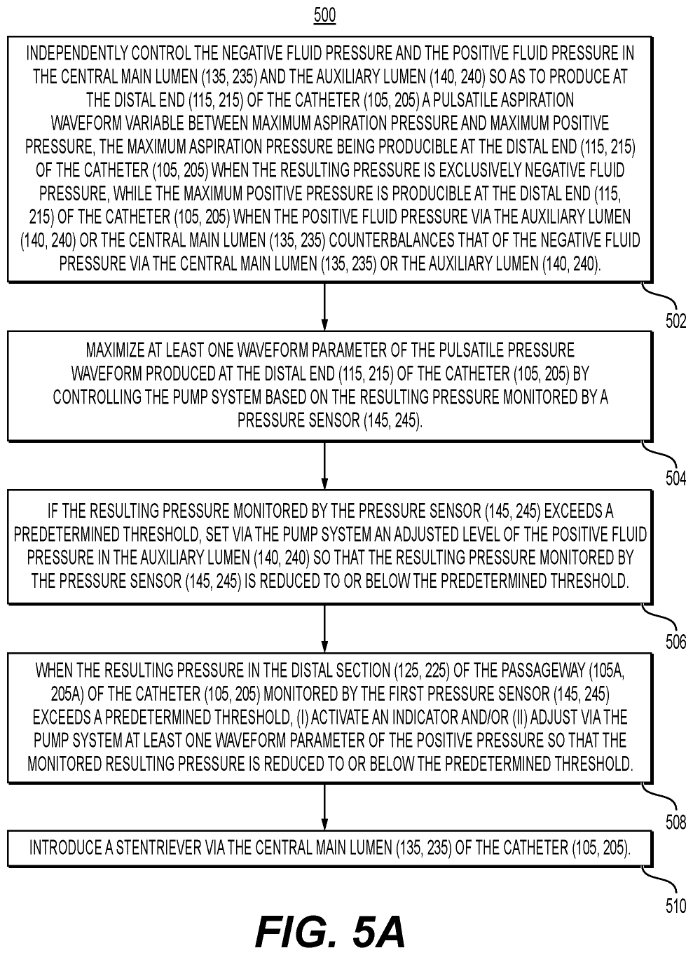

As shown by the dashed lines in FIG. 1A, a distal end of the central main lumen 135 and a distal end of the auxiliary lumen 140 are in fluid communication with one another where the negative fluid pressure and the positive fluid pressure combine as a resulting pressure (see the waveform in FIG. 4) in the single lumen at the distal section 143 of the passageway 105A.

As alluded to above, and with reference to FIG. 3, a pulsatile pump system is in fluid communication with the catheter 105 to independently control the negative (aspiration suction) fluid pressure in the central main lumen 135 and the positive fluid pressure in the auxiliary lumen 140. It will be appreciated by those skilled in the art that, while at the very least intermittently, continuous pressure in one lumen does not necessarily need to be provided to oppose the pressure in the other lumen (discussed in greater detail below). The pump system includes a programmable controller 183, tubing 175, a fluid collection chamber/canister 180, and a proximal hub 160. The controller 183 includes a positive pressure source (e.g., a positive pressure pump or a pressurized fluid reservoir with an on/off valve) with a pressure exchange PX port and a vacuum pump with vacuuming VAC port. In some examples, the proximal hub 160 is a dual side port proximal hub with a negative fluid pressure port 165 and a positive fluid pressure port 170. The negative fluid pressure port 165 is in fluid communication with the central main lumen 135 and the positive fluid pressure port 170 is in fluid communication with the auxiliary lumen 140.

FIG. 3 illustrates a schematic representation of the vacuum pump combined into a circuit with the positive pressure source. This circuit is then connected to the proximal end of the catheter 105 via the negative fluid pressure port 165 and the positive fluid pressure port 170. A fluid collection container 180 within the aspiration circuit collects all the content aspirated from the patient's body. In some examples, the controller's vacuum source is operable to evacuate the fluid collection container 180.

The tubing 175 in the aspiration circuit as well as the tubing 175 in the positive pressure circuit is joined to the proximal end of the catheter 105 via the respective ports 165, 170 on the proximal hub 160. In the aspiration circuit, the tubing 175 is discontinuous to provide for the fluid collection container 180, whereas, in the positive pressure circuit, the tubing 175 can be continuous from the positive pressure port 170 to the PX port on the controller.

As exemplified by the waveform in FIG. 4, the controller 183 independently controls the positive pressure source and the vacuum pump so as to produce, at the distal end 115 of the catheter 105, a resulting pressure with a pulsatile aspiration waveform variable between a maximum aspiration pressure (see the valleys of the waveform in FIG. 4) and a maximum positive pressure (see the peaks of the waveform in FIG. 4).

It is noted that the “0” on the y-axis could be atmospheric pressure, or it could be systolic or diastolic blood pressure of the patient. It will be appreciated that the peak maximum pressure can be set to be above or below this line. Moreover, it can be set below a specific pressure (e.g., approximately 50 Kpa above blood pressure of the patient) that is determined to be safe or critical.

The maximum aspiration pressure is producible at the distal end 115 of the catheter 105 when the resulting pressure is exclusively negative fluid pressure via the central main lumen 135. In this scenario, the positive pressure source does not inject any fluid to cause an increase in pressure. Similarly, the maximum positive pressure is producible at the distal end 115 of the catheter 105 when the controller 183 controls the positive pressure source to inject a biocompatible fluid (e.g., saline) via the auxiliary lumen 140. In some examples, the biocompatible fluid is injected into the auxiliary lumen 140 in a direction generally parallel to the longitudinal axis 60. In other examples, the biocompatible fluid can be injected at an angle (e.g., between 0 to 90 degrees) relative to the longitudinal axis. The maximum positive pressure (of the resulting pressure) can be produced by the positive fluid pressure from the positive pressure source at least partially counterbalancing the negative fluid pressure from the vacuum pump.

As alluded to above, in other examples, the positive pressure source with the pressure exchange PX port and the vacuum pump with the vacuuming VAC port can be reversed with the central main lumen 135 and the auxiliary lumen 140, resulting in negative fluid pressure being produced in the auxiliary lumen 140 and positive fluid pressure being produced in the central main lumen 135.

In some examples, the maximum positive pressure is less than ambient atmospheric pressure. In other examples, the maximum positive pressure is approximately ambient atmospheric pressure. In even further examples, the maximum positive pressure can exceed ambient atmospheric pressure without departing from the spirit and scope of the present disclosure. By way of example, the maximum positive pressure can exceed atmospheric pressure but be less than diastolic blood pressure of the patient, or it can fall between diastolic blood pressure and systolic blood pressure of the patient, or it can exceed systolic blood pressure of the patient.

In order to monitor the aforementioned resulting pressure at the distal tip of the catheter 105, a pressure sensor 145 is arranged on an inner surface of the outer wall of the distal section 125 of the catheter 105. The pressure sensor 145 monitors the resulting pressure in the distal section 143 of the passageway 105A of the catheter 105. By its monitoring and providing a feedback loop with the controller 183, at least one waveform parameter of the maximum pulsatile aspiration waveform (see FIG. 4) produced at the distal end 115 of the catheter 105 can be maximized (in regard to positive pressure and/or negative pressure). This is done by controlling the controller 183 based on the resulting pressure monitored by the pressure sensor 145, the controller 183 being operable to adjust the rate, volume, and/or frequency of the positive pressure injection of fluid through the auxiliary lumen 140.

Additionally, when the resulting pressure in the distal section of the passageway of the catheter monitored by the pressure sensor 145 exceeds a predetermined threshold the system 100 is operable to perform a number of functions. For example, an indicator can be activated to alert the physician to the pressure reading and/or controller 183 is controllable to adjust the positive pressure fluid via the auxiliary lumen 140 (by varying the volume of biocompatible fluid injected) so that the monitored resulting pressure is returned to or below the predetermined threshold. By way of example, the predetermined threshold could be ambient atmospheric pressure, the diastolic blood pressure of the patient, the systolic blood pressure of the patient, etc.

By implementing the pulsatile aspiration system 100 in this manner, cycling between the maximum aspiration pressure and the maximum positive pressure in the distal section 125 of the catheter 105 can be done as a very high frequency with only a small amount of biocompatible fluid injection required. With the dual lumen 135, 140 structure keeping the negative pressure and the positive pressure pulse separate, the dampening effect on the pulsatile aspiration waveform (FIG. 4) can be minimized since the negative pressure in the central main lumen 135 does not need to change significantly, thereby enabling the aforementioned rapid pressure changes in the distal section 125.

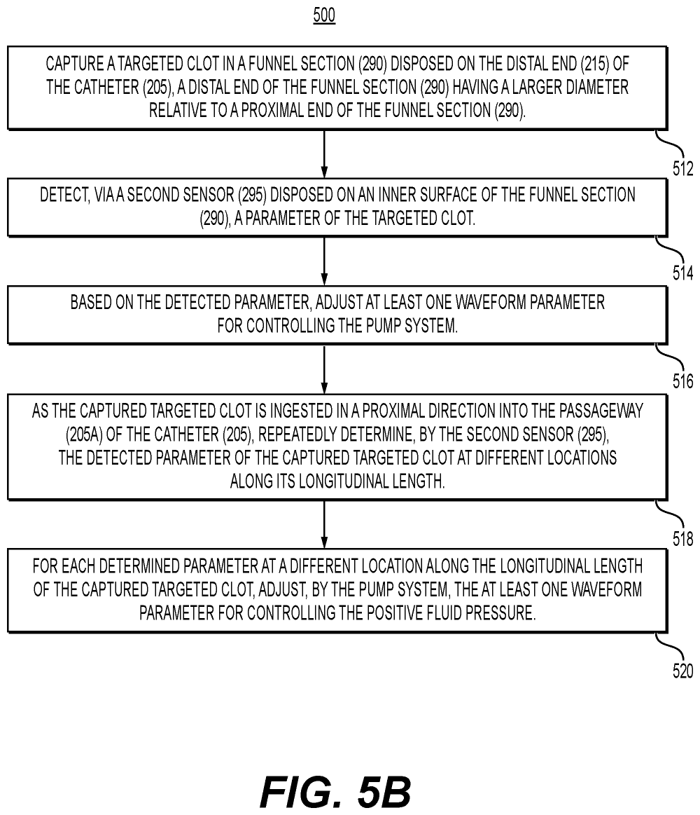

Further to the above, and in accordance with the presently disclosed technology, the above-described pulsatile aspiration system 100 enables performance of the following method 500, as shown in FIG. 5A.

The negative fluid pressure is independently controlled in one of the central main lumen 135 the auxiliary lumen 140 and the positive fluid pressure in the auxiliary lumen 140 so as to produce at the distal end 115 of the catheter 105 a maximum pulsatile aspiration waveform variable between a maximum aspiration pressure and a maximum positive pressure (Step 502). The maximum aspiration pressure is producible at the distal end 115 of the catheter 105 when the resulting pressure is exclusively negative fluid pressure, while the maximum fluid pressure is producible at the distal end 115 of the catheter 105 when the positive fluid pressure (via the auxiliary lumen 140 or the central main lumen 135, as discussed above) counterbalances that of the negative fluid pressure (via the other of the central main lumen 135 and the auxiliary lumen 135, as discussed above).

The method 500 further includes the step 504 of maximizing at least one waveform parameter of the pulsatile pressure waveform produced at the distal end 115 of the catheter 105 by controlling the pump system based on the resulting pressure monitored by a pressure sensor 145.

If the resulting pressure monitored by the pressure sensor 145 exceeds a predetermined threshold (e.g., ambient pressure, diastolic or systolic blood pressure, etc., as discussed above), the method 500 further includes the step 506 of setting, via the pump system, an adjusted level of the positive fluid pressure (in the auxiliary lumen 140 or, in other examples, the central main lumen 135) so that the resulting pressure monitored by the pressure sensor 145 is reduced to or below the predetermined threshold.

Similarly, when the resulting pressure in the distal section 125 of the passageway 105A of the catheter 105 monitored by the pressure sensor 145 exceeds the predetermined threshold, the method 500 further includes the step 508 of activating an indicator and/or (ii) adjusting, by the pump system, the at least one waveform parameter of the positive pressure fluid (via the auxiliary lumen 140 of the central main lumen 135, as discussed above) so that the monitored resulting pressure is reduced to or below the predetermined threshold.

The method 500 further includes the step 510 of introducing a stentriever via the central main lumen 135 of the catheter 105.

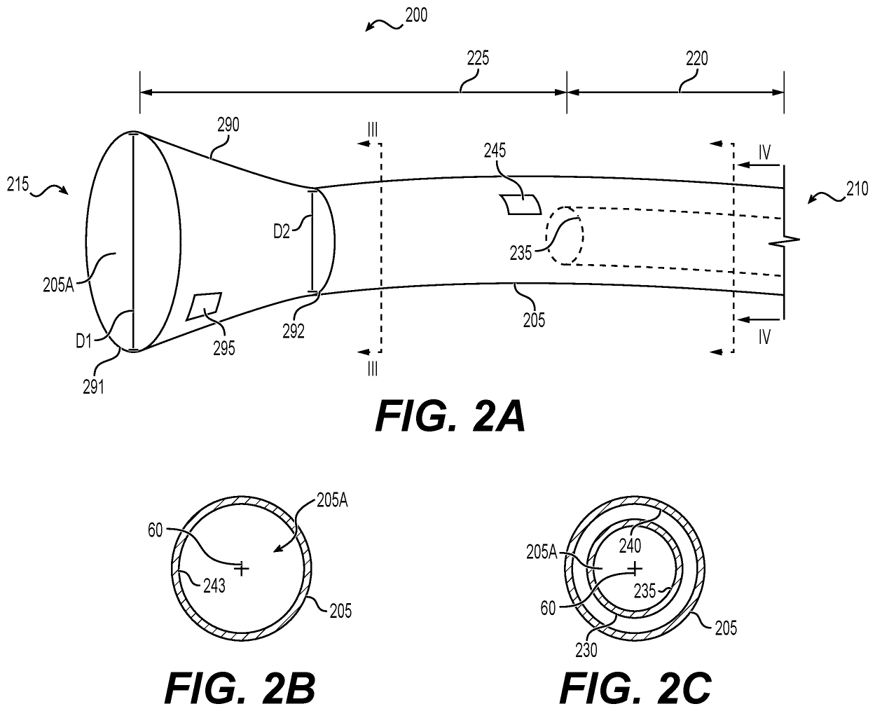

FIG. 2A depicts a perspective view of another example of a distal tip of a catheter of a pulsatile aspiration system. FIG. 2B depicts a cross-sectional view of the distal tip of FIG. 2A, taken along line III in FIG. 2A. FIG. 2C depicts another cross-sectional view of the distal tip of FIG. 2A, taken along line IV in FIG. 2A. FIG. 3, while illustrating the distal tip of FIG. 1A, is representative of the pulsatile aspiration system 200 description that follows.

With reference now to FIGS. 2A-2C and 3, a distal tip of a catheter 205 is shown, which forms a portion of a pulsatile aspiration system 200 (FIG. 3). The catheter 205 has an outer wall that extends in a longitudinal direction along a longitudinal axis 60 from a proximal end 210 to an opposite free distal end 215, with a passageway 205A being defined therethrough. Referring to FIG. 2A in conjunction with FIG. 3, and like the previously described example, the proximal end 210 is connected to a hub 160, which is described in greater detail below. The catheter 205 further includes a proximal section 220 that is associated with a proximal section 235, 240 of the passageway 205A and a distal section 225 disposed distally thereof that is associated with a distal section 243 of the passageway 205A and terminates at the distal end 215. In some examples, the distal section 225 can be varied in length between approximately 0 centimeters (where the distal section 225 is essentially an extension of the proximal section 220, and the proximal section 220 extends approximately to the free distal end 215, but optimally at least a few millimeters therefrom) and 5 centimeters. In some examples, like the previously described one, the distal section 225 can include a radiopaque marker (not shown) for tracking a location of the distal tip in the patient's body.

As particularly seen in FIG. 2C, the proximal section 235, 240 of the passageway 205A includes an internal wall 230 that extends in the longitudinal direction through the passageway 105A. The internal wall 230 divides the proximal section 235, 240 of the passageway 205A into a central main lumen 235 and an auxiliary lumen 240, through both of which fluid is receivable. The central main lumen 235 is at least partially defined by the internal wall 230 such that it is defined on one side of the internal wall 230 (e.g., radially inward towards the longitudinal axis 60 relative to the internal wall 230) and is sized so as to be capable of receiving a stentriever. The auxiliary lumen 240 is at least partially defined by the internal wall such that it is arranged on another side of the internal wall 230 (e.g., radially outwards from the longitudinal axis 60 relative to the internal wall 230). The internal wall 230 is designed such that, in cross-section, the areas of central main lumen 235 and auxiliary lumen 240 have a predetermined ratio relative to one another to maximize clot ingestion. Depending on the lumens used for negative fluid pressure and positive fluid pressure, respectively (and discussed in greater detail below), the ratio of negative fluid pressure lumen to positive pressure lumen area can be at least 70:30, such as 90:10 or 95:5. In contrast, and as particularly seen in FIG. 2B, the distal section 243 of the passageway 205A is undivided. However, as alluded to above, it will be appreciated that, in some examples, the distal section 225/243 (as shown in the figures) can be configured essentially the same as the proximal section 220 by extending the internal wall 230 all the way to the free distal end 215.

In this example, the central main lumen 235 and auxiliary lumen 240 are arranged concentrically relative to the longitudinal axis 60. More specifically, in this example, the internal wall 230 is an inner catheter 230 arranged concentrically within the proximal section 220 of the passageway 205A of the catheter 205 and is slidable in the longitudinal direction independently of the catheter 205 (i.e., independently of the auxiliary lumen 240). However, it will be appreciated that, in other examples, the central main lumen 235 can be arranged eccentrically/offset relative to the longitudinal axis 60. The inner catheter 230 has a channel in the longitudinal direction defined therein serving as the central main lumen 235 of the proximal section 220 of the passageway 205A of the catheter 205 while a region defined in a radial direction between the inner catheter 230 and the proximal section 220 of the catheter 205 serves as the auxiliary lumen 240.

In some examples, in the proximal section 220, the central main lumen 235 produces negative fluid pressure therein by way of a fluid (e.g., blood) and pump system that is described in greater detail below. In these examples, the auxiliary lumen 240 produces positive fluid pressure therein by way of a biocompatible fluid and pump system that is described in greater detail below.

It will be appreciated that, in other examples and in accordance with the presently disclosed technology, the positive and negative pressures can be reversed such that negative fluid pressure is produced in the auxiliary lumen 240 and positive fluid pressure is produced in the main lumen 235. In the example shown in FIG. 2A, this may be particularly advantageous because the clot ingestion volume can be maximized by providing the aspiration along the inner circumference of the catheter 205.

As shown by the dashed lines in FIG. 2A, a distal end of the central main lumen 235 and a distal end of the auxiliary lumen 240 are in fluid communication with one another where the negative fluid pressure and the positive fluid pressure combine as a resulting pressure (see the waveform in FIG. 4) in the single lumen at the distal section 243 of the passageway 205A.

As alluded to above, and with reference to FIG. 3, a pulsatile pump system is in fluid communication with the catheter 205 to independently control the negative (aspiration suction) fluid pressure in the central main lumen 235 and the positive fluid pressure in the auxiliary lumen 240. It will be appreciated by those skilled in the art that, while at the very least intermittently, continuous pressure in one lumen does not necessarily need to be provided to oppose the pressure in the other lumen (discussed in greater detail below). The pump system includes a programmable controller 183, tubing 175, a fluid collection chamber 180, and a proximal hub 160. The controller 183 includes a positive pressure source (e.g., a positive pressure pump or a pressurized fluid reservoir with an on/off valve) with a pressure exchange PX port and a vacuum pump with vacuuming VAC port. In some examples, the proximal hub 160 is a dual side port proximal hub with a negative fluid pressure port 165 and a positive fluid pressure port 170. The negative fluid pressure port 165 is in fluid communication with the central main lumen 235 and the positive fluid pressure port 170 is in fluid communication with the auxiliary lumen 240.

As discussed above, FIG. 3 illustrates a schematic representation of the vacuum pump combined into a circuit with the positive pressure source. This circuit is then connected to the proximal end of the catheter 205 via the negative fluid pressure port 165 and the positive fluid pressure port 170. A fluid collection container 180 within the aspiration circuit collects all the content aspirated from the patient's body.

The tubing 175 in the aspiration circuit as well as the tubing 175 in the positive pressure circuit is joined to the proximal end of the catheter 205 via the respective ports 165, 170 on the proximal hub 160. In the aspiration circuit, the tubing 175 is discontinuous to provide for the fluid collection container 180, whereas, in the positive pressure circuit, the tubing 175 can be continuous from the positive pressure port 170 to the PX port on the controller.

As exemplified by the waveform in FIG. 4, the controller 183 independently controls the positive pressure source and the vacuum pump so as to produce, at the distal end 215 of the catheter 205, a resulting pressure with a pulsatile aspiration waveform variable between a maximum aspiration pressure (see the valleys of the waveform in FIG. 4) and a maximum positive pressure (see the peaks of the waveform in FIG. 4).

The maximum aspiration pressure is producible at the distal end 215 of the catheter 205 when the resulting pressure is exclusively negative fluid pressure via the central main lumen 235. In this scenario, the positive pressure source does not inject any fluid to cause an increase in pressure. Similarly, the maximum positive pressure is producible at the distal end 215 of the catheter 205 when the controller 183 controls the positive pressure source to inject a biocompatible fluid (e.g., saline) via the auxiliary lumen 240. In some examples, the biocompatible fluid is injected into the auxiliary lumen 140 in a direction generally parallel to the longitudinal axis 60. In other examples, the biocompatible fluid can be injected at an angle (c.g., between 0 to 90 degrees) relative to the longitudinal axis. The maximum positive pressure (of the resulting pressure) can be produced by the positive fluid pressure from the positive pressure source at least partially counterbalancing the negative fluid pressure from the vacuum pump.

As alluded to above, in other examples, the positive pressure source with the pressure exchange PX port and the vacuum pump with the vacuuming VAC port can be reversed with the central main lumen 235 and the auxiliary lumen 240, resulting in negative fluid pressure being produced in the auxiliary lumen 240 and positive fluid pressure being produced in the central main lumen 235.

In some examples, the maximum positive pressure is less than ambient atmospheric pressure. In other examples, the maximum positive pressure is approximately ambient atmospheric pressure. In even further examples, the maximum positive pressure can exceed ambient atmospheric pressure without departing from the spirit and scope of the present disclosure. By way of example, the maximum positive pressure can exceed atmospheric pressure but be less than diastolic blood pressure of the patient, or it can fall between diastolic blood pressure and systolic blood pressure of the patient, or it can exceed systolic blood pressure of the patient.

In order to monitor the aforementioned resulting pressure at the distal tip of the catheter 205, a pressure sensor 245 is arranged on an inner surface of the outer wall of the distal section 225 of the catheter 205. The pressure sensor 245 monitors the resulting pressure in the distal section 243 of the passageway 205A of the catheter 205. By its monitoring and providing a feedback loop with the controller 183, at least one waveform parameter of the maximum pulsatile aspiration waveform (see FIG. 4) produced at the distal end 215 of the catheter 205 can be maximized (in regard to positive pressure and/or negative pressure). This is done by controlling the controller 183 based on the resulting pressure monitored by the pressure sensor 245, the controller 183 being operable to adjust the rate, volume, and/or frequency of the positive pressure injection of fluid through the auxiliary lumen 240.

Additionally, when the resulting pressure in the distal section of the passageway of the catheter monitored by the pressure sensor 245 exceeds a predetermined threshold the system 200 is operable to perform a number of functions. For example, an indicator can be activated to alert the physician to the pressure reading and/or controller 183 is controllable to adjust the positive pressure fluid via the auxiliary lumen 240 (by varying the volume of biocompatible fluid injected) so that the monitored resulting pressure is returned to or below the predetermined threshold. By way of example, the predetermined threshold could be ambient atmospheric pressure, the diastolic blood pressure of the patient, the systolic blood pressure of the patient, etc.

Moreover, the example depicted in FIG. 2A can include, in the distal section 225 of the catheter 205, a funnel section 290 that has a free distal end/mouth 291 and an opposite proximal end 292 attached to a distal portion of the catheter 205. As shown, the free distal end 291 of the funnel section 290 has a larger diameter DI relative a diameter D2 of the proximal end 292 of the funnel section 290 in order to receive a clot. Disposed on an inner surface of the funnel section 290 is a second sensor 295. The second sensor 295 detects a parameter of the clot (e.g., dependent on fibrin content, RBC content, or impedance) of the clot capturable in the funnel section 290. By way of example, the second sensor 295 could be an impedance-based sensor or an optical sensor using visible or near-infrared light. In other examples, the second sensor 295 could be a second pressure sensor that provides supplemental feedback to the controller 183 and can be used (similar to the pressure sensor 245) to adjust at least one waveform parameter for controlling the pump system. As those skilled in the art will appreciate, while the funnel section 290 and second sensor 295 are described with respect to the example of FIGS. 2A-2C, it, of course, can be employed in the first-described example of FIGS. 1A-1C without departing from the spirit and scope of the present disclosure.

By implementing the pulsatile aspiration system 200 in this manner, cycling between the maximum aspiration pressure and the maximum positive pressure in the distal section 225 of the catheter 105 can be done as a very high frequency with only a small amount of biocompatible fluid injection required. With the dual lumen 235, 240 structure keeping the negative pressure and the positive pressure pulse separate, the dampening effect on the pulsatile aspiration waveform (FIG. 4) can be minimized since the negative pressure in the central main lumen 235 does not need to change significantly, thereby enabling the aforementioned rapid pressure changes in the distal section 225.

Further to the above, and in accordance with the presently disclosed technology, the above-described pulsatile aspiration system 200 enables performance of the following method 500, as shown in FIGS. 5A and 5B.

The negative air pressure is independently controlled in one of the central main lumen 235 the auxiliary lumen 240 and the positive fluid pressure in the auxiliary lumen 240 so as to produce at the distal end 215 of the catheter 205 a maximum pulsatile aspiration waveform variable between a maximum aspiration pressure and a maximum positive pressure (Step 502). The maximum aspiration pressure is producible at the distal end 215 of the catheter 205 when the resulting pressure is exclusively negative fluid pressure, while the maximum fluid pressure is producible at the distal end 215 of the catheter 205 when the positive fluid pressure (via the auxiliary lumen 240 or the central main lumen 235, as discussed above) counterbalances that of the negative fluid pressure (via the other of the central main lumen 235 and the auxiliary lumen 135, as discussed above).

The method 500 further includes the step 504 of maximizing at least one waveform parameter of the pulsatile pressure waveform produced at the distal end 215 of the catheter 205 by controlling the pump system based on the resulting pressure monitored by a pressure sensor 245.

If the resulting pressure monitored by the pressure sensor 245 exceeds a predetermined threshold (e.g., ambient pressure, diastolic or systolic blood pressure, etc., as discussed above), the method 500 further includes the step 506 of setting, via the pump system, an adjusted level of the positive fluid pressure (in the auxiliary lumen 240 or, in other examples, the central main lumen 235) so that the resulting pressure monitored by the pressure sensor 245 is reduced to or below the predetermined threshold.

Similarly, when the resulting pressure in the distal section 225 of the passageway 205A of the catheter 205 monitored by the pressure sensor 245 exceeds the predetermined threshold, the method 500 further includes the step 508 of activating an indicator and/or (ii) adjusting, by the pump system, the at least one waveform parameter of the positive pressure fluid (via the auxiliary lumen 240 of the central main lumen 235, as discussed above) so that the monitored resulting pressure is reduced to or below the predetermined threshold.

The method 500 further includes the step 510 of introducing a stentriever via the central main lumen 235 of the catheter 205.

Referring to FIG. 5B, the method 500 further includes the step 512 of capturing a targeted clot in a funnel section 290 disposed on the distal end 215 of the catheter 205, a distal end 291 of the funnel section 290 having a larger diameter DI relative to a diameter D2 of the proximal end 292 of the funnel section 290.

The method 500 further includes the step 514 of detecting, via a second sensor 295 disposed on an inner surface of the funnel section 290, a parameter of the targeted clot. By way of non-limiting example, the parameter can be a mechanical property of the clot, such as fibrin content, RBC content, clot impedance, and/or combinations thereof.

The method 500 further includes the step 516 of adjusting, based on the detected parameter of the captured targeted clot, at least one waveform parameter for controlling the pump system.

The method 500 further includes the step 518 of, as the captured targeted clot is ingested in a proximal direction into the passageway 205A of the catheter 205, repeatedly determining, by the second sensor 295, the detected parameter of the captured targeted clot at different locations along its longitudinal length.

In some examples, the second sensor 295 can repeatedly determine other parameters (not necessarily measured properties of the clot) to determine when and how much of the clot has been ingested. This information can, for example, be used to switch from cyclic to continuous aspiration and vice versa.

The method 500 further includes the step 520 of, for each determined parameter at a different location along the longitudinal length of the captured targeted clot, adjusting, by the pump system, the at least one waveform parameter for controlling the positive fluid pressure.

The disclosed technology described herein can be further understood according to the following clauses:

Clause 1. A pulsatile aspiration system comprising: a catheter comprising: an outer wall extending in a longitudinal direction from a proximal end to an opposite distal end defining a passageway therethrough; a proximal section with an associated proximal section of the passageway; a distal section disposed distally thereof with an associated distal section of the passageway; and an internal wall extending in the longitudinal direction therethrough and dividing the proximal section of the passageway into (i) a central main lumen defined radially inward relative to the internal wall and (ii) an auxiliary lumen at least partially defined by the internal wall and internal to the outer wall, the central main lumen, through which a first fluid is receivable, being configured to produce one of negative fluid pressure or positive fluid pressure therein, the auxiliary lumen, through which a second fluid is receivable, being configured to produce the other of the positive fluid pressure or the negative fluid pressure therein, and a distal portion of the central main lumen and a distal portion of the auxiliary lumen are in fluid communication with one another where the negative fluid pressure and the positive fluid pressure combine as a resulting pressure in the distal section of the passageway; and a pump system in fluid communication with the catheter and independently controlling the negative fluid pressure and the positive fluid pressure in the central main lumen and the auxiliary lumen so as to produce at the distal end of the catheter a pulsatile aspiration waveform variable between a maximum aspiration pressure and a maximum positive pressure, the maximum aspiration pressure being producible at the distal end of the catheter when the resulting pressure is exclusively negative fluid pressure, and the maximum positive pressure being producible at the distal end of the catheter when the positive fluid pressure counterbalances that of the negative fluid pressure.

Clause 2. The pulsatile aspiration system in accordance with clause 1, wherein the auxiliary lumen is arranged eccentrically of the central main lumen.

Clause 3. The pulsatile aspiration system in accordance with clause 2, wherein the internal wall is permanently fixed in position within and dividing the proximal section of the passageway of the catheter into the central main lumen and the auxiliary lumen on opposite sides of the internal wall in a radial direction.

Clause 4. The pulsatile aspiration system in accordance with clause 1, wherein the central main lumen and auxiliary lumen are arranged concentrically.

Clause 5. The pulsatile aspiration system in accordance with clause 1, wherein the internal wall is an inner catheter arranged within the proximal section of the passageway of the catheter and slidable in the longitudinal direction independently of the catheter; the inner catheter having a channel in the longitudinal direction defined therein serving as the central main lumen of the proximal section of the passageway of the catheter while a region defined in a radial direction between the inner catheter and the outer wall of proximal section of the catheter serves as the auxiliary lumen.

Clause 6. The pulsatile aspiration system in accordance with any one of clauses 1-5, further comprising a pressure sensor arranged on an inner surface of the outer wall of the distal section of the catheter, the pressure sensor monitoring the resulting pressure in the distal section of the passageway of the catheter; wherein at least one parameter of the pulsatile aspiration waveform produced at the distal end of the catheter is adjusted by controlling the pump system based on the resulting pressure monitored by the pressure sensor.

Clause 7. The pulsatile aspiration system in accordance with clause 6, wherein

when the resulting pressure in the distal section of the passageway of the catheter monitored by the pressure sensor exceeds a predetermined threshold: (i) an indicator is activatable;

and/or (ii) the pump system is controllable to adjust the positive pressure fluid via the central main lumen or the auxiliary lumen so that the monitored resulting pressure is reduced to or below the predetermined threshold.

Clause 8. The pulsatile aspiration system in accordance with any one of clauses 1-7, further comprising: a funnel section having a free distal end and an opposite proximal end attached to the distal end of the catheter; wherein the free distal end of the funnel section has a larger diameter relative to the proximal end of the funnel section; and a second sensor disposed on an inner surface of the funnel section detecting a parameter of a clot associated with fibrin content of the clot, the clot being capturable in the funnel section.

Clause 9. The pulsatile aspiration system in accordance with any one of clauses 1-8, wherein a stentriever is receivable in the central main lumen of the catheter.

Clause 10. The pulsatile aspiration system in accordance with any one of clauses 1-9, wherein the distal section of the passageway is undivided.

Clause 11. A method for operating a pulsatile aspiration system including a catheter having an outer wall extending in a longitudinal direction from a proximal end to an opposite distal end and defining a passageway therethrough; the catheter including a proximal section with an associated proximal section of the passageway and a distal section disposed distally thereof with an associated distal section of the passageway; the proximal section of the passageway is divided by an internal wall extending in the longitudinal direction therethrough into a central main lumen defined radially inward relative to the internal wall and an auxiliary at least partially defined by the internal wall; the central main lumen through which a first fluid is receivable being configured to produce one of negative fluid pressure or positive fluid pressure therein, while the auxiliary lumen through which a second fluid is receivable being configured to produce the other of the negative fluid pressure or the positive fluid pressure therein; wherein a distal end of the central main lumen and a distal end of the auxiliary lumen are in fluid communication with one another where the negative fluid pressure and the positive fluid pressure combine as a resulting pressure in the distal section of the passageway; the pulsatile aspiration system further including a pump system in fluid communication with the catheter; the method comprising the step of: independently controlling the negative fluid pressure and the positive fluid pressure in the central main lumen and the auxiliary lumen so as to produce at the distal end of the catheter a pulsatile aspiration waveform variable between maximum aspiration pressure and a maximum positive pressure; the maximum aspiration pressure being producible at the distal end of the catheter when the resulting pressure is exclusively negative fluid pressure, while the maximum positive pressure being producible at the distal end of the catheter when the positive fluid pressure counterbalances that of the negative fluid pressure.

Clause 12. The method in accordance with clause 11, further comprising the step of maximizing at least one waveform parameter of the pulsatile pressure waveform produced at the distal end of the catheter by controlling the pump system based on the resulting pressure monitored by a pressure sensor.

Clause 13. The method in accordance with clause 12, further comprising the step of: if the resulting pressure monitored by the pressure sensor exceeds a predetermined threshold, setting, via the pump system, an adjusted level of the positive fluid pressure so that the resulting pressure monitored by the pressure sensor is reduced to or below the predetermined threshold.

Clause 14. The method in accordance with any one of clauses 11-13, further comprising the steps of: capturing a targeted clot in a funnel section disposed on the distal end of the catheter, a distal end of the funnel section having a larger diameter relative to a proximal end of the funnel section; detecting, via a second sensor disposed on an inner surface of the funnel section, a parameter of the targeted clot; and based the detected parameter, adjusting at least one waveform parameter for controlling the pump system.

Clause 15. The method in accordance with any one of clauses 11-14, further comprising arranging the auxiliary lumen eccentrically of the central main lumen.

Clause 16. The method in accordance with clause 15, further comprising permanently fixing the internal wall in position within and dividing the proximal section of the passageway of the catheter into the central main lumen and the auxiliary lumen on opposite sides of the internal wall in a radial direction.

Clause 17. The method in accordance with any one of clauses 11-14, further comprising concentrically arranging the central main lumen and auxiliary lumen.

Clause 18. The method in accordance with clause 17, further comprising arranging the internal wall as an inner catheter within the proximal section of the passageway of the catheter and slidable in the longitudinal direction independently of the catheter; and forming a channel in the inner catheter in the longitudinal direction defined therein serving as the central main lumen of the proximal section of the passageway of the catheter while a region defined in a radial direction between the inner catheter and the proximal section of the catheter serves as the auxiliary lumen.

Clause 19. The method in accordance with any one of clauses 11-18, wherein when the resulting pressure in the distal section of the passageway of the catheter monitored by the pressure sensor exceeds a predetermined threshold, further comprising the step of: (i) activating an indicator; and/or (ii) adjusting, via the pump system, at least one waveform parameter of the positive pressure fluid so that the monitored resulting pressure is below the predetermined threshold.

Clause 20. The method in accordance with any one of clauses 11-19, further comprising the step of introducing a stentriever via the central main lumen of the catheter.

Clause 21. The method in accordance with clause 14, wherein as the captured targeted clot is ingested in a proximal direction into the passageway of the catheter, the second sensor repeatedly determines the detected parameter of the captured targeted clot at different locations along its longitudinal length; and for each determined parameter at a different location along the longitudinal length of the captured targeted clot, the method further comprises the step of adjusting the at least one waveform parameter for controlling the positive fluid pressure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

The descriptions contained herein are examples of the disclosed technology and are not intended in any way to limit the scope of the disclosed technology. As described herein, the disclosed technology contemplates many variations and modifications of pulse generation mechanism for producing a pulsatile aspiration pressure waveform using a static vacuum source. Modifications and variations apparent to those having skilled in the pertinent art according to the teachings of this disclosure are intended to be within the scope of the claims which follow.

What is claimed is:

1. A pulsatile aspiration system comprising:
a catheter comprising:
an outer wall extending in a longitudinal direction from a proximal end to an opposite distal end defining a passageway therethrough;
a proximal section with an associated proximal section of the passageway;
a distal section disposed distally thereof with an associated distal section of the passageway; and
an internal wall extending in the longitudinal direction therethrough and dividing the proximal section of the passageway into (i) a central main lumen defined radially inward relative to the internal wall and (ii) an auxiliary lumen at least partially defined by the internal wall and internal to the outer wall,
the central main lumen, through which a first fluid is receivable, being configured to produce one of negative fluid pressure or positive fluid pressure therein,
the auxiliary lumen, through which a second fluid is receivable, being configured to produce the other of the positive fluid pressure or the negative fluid pressure therein, and
a distal portion of the central main lumen and a distal portion of the auxiliary lumen are in fluid communication with one another where the negative fluid pressure and the positive fluid pressure combine as a resulting pressure in the distal section of the passageway; and
a pump system in fluid communication with the catheter and independently controlling the negative fluid pressure and the positive fluid pressure in the central main lumen and the auxiliary lumen so as to produce at the distal end of the catheter a pulsatile aspiration waveform variable between a maximum aspiration pressure and a maximum positive pressure,
the maximum aspiration pressure being producible at the distal end of the catheter when the resulting pressure is exclusively negative fluid pressure, and
the maximum positive pressure being producible at the distal end of the catheter when the positive fluid pressure counterbalances that of the negative fluid pressure.

2. The pulsatile aspiration system in accordance with claim 1, wherein the auxiliary lumen is arranged eccentrically of the central main lumen.

3. The pulsatile aspiration system in accordance with claim 2, wherein the internal wall is permanently fixed in position within and dividing the proximal section of the passageway of the catheter into the central main lumen and the auxiliary lumen on opposite sides of the internal wall in a radial direction.

4. The pulsatile aspiration system in accordance with claim 1, wherein the central main lumen and auxiliary lumen are arranged concentrically.

5. The pulsatile aspiration system in accordance with claim 1, wherein the internal wall is an inner catheter arranged within the proximal section of the passageway of the catheter and slidable in the longitudinal direction independently of the catheter; the inner catheter having a channel in the longitudinal direction defined therein serving as the central main lumen of the proximal section of the passageway of the catheter while a region defined in a radial direction between the inner catheter and the outer wall of proximal section of the catheter serves as the auxiliary lumen.

6. The pulsatile aspiration system in accordance with claim 1, further comprising a pressure sensor arranged on an inner surface of the outer wall of the distal section of the catheter, the pressure sensor monitoring the resulting pressure in the distal section of the passageway of the catheter;
wherein at least one parameter of the pulsatile aspiration waveform produced at the distal end of the catheter is adjusted by controlling the pump system based on the resulting pressure monitored by the pressure sensor.

7. The pulsatile aspiration system in accordance with claim 6, wherein when the resulting pressure in the distal section of the passageway of the catheter monitored by the pressure sensor exceeds a predetermined threshold: (i) an indicator is activatable; and/or (ii) the pump system is controllable to adjust the positive pressure fluid via the central main lumen or the auxiliary lumen so that the monitored resulting pressure is reduced to or below the predetermined threshold.

8. The pulsatile aspiration system in accordance with claim 1, further comprising:
a funnel section having a free distal end and an opposite proximal end attached to the distal end of the catheter; wherein the free distal end of the funnel section has a larger diameter relative to the proximal end of the funnel section; and
a second sensor disposed on an inner surface of the funnel section detecting a parameter of a clot associated with fibrin content of the clot, the clot being capturable in the funnel section.

9. The pulsatile aspiration system in accordance with claim 1, wherein a stentriever is receivable in the central main lumen of the catheter.

10. The pulsatile aspiration system in accordance with claim 1, wherein the distal section of the passageway is undivided.

11. A method for operating a pulsatile aspiration system including a catheter having an outer wall extending in a longitudinal direction from a proximal end to an opposite distal end and defining a passageway therethrough; the catheter including a proximal section with an associated proximal section of the passageway and a distal section disposed distally thereof with an associated distal section of the passageway; the proximal section of the passageway is divided by an internal wall extending in the longitudinal direction therethrough into a central main lumen defined radially inward relative to the internal wall and an auxiliary lumen at least partially defined by the internal wall; the central main lumen through which a first fluid is receivable being configured to produce one of negative fluid pressure or positive fluid pressure therein, while the auxiliary lumen through which a second fluid is receivable being configured to produce the other of the negative fluid pressure or the positive fluid pressure therein; wherein a distal end of the central main lumen and a distal end of the auxiliary lumen are in fluid communication with one another where the negative fluid pressure and the positive fluid pressure combine as a resulting pressure in the distal section of the passageway; the pulsatile aspiration system further including a pump system in fluid communication with the catheter; the method comprising the step of:
independently controlling the negative fluid pressure and the positive fluid pressure in the central main lumen and the auxiliary lumen so as to produce at the distal end of the catheter a pulsatile aspiration waveform variable between maximum aspiration pressure and a maximum positive pressure; the maximum aspiration pressure being producible at the distal end of the catheter when the resulting pressure is exclusively negative fluid pressure, while the maximum positive pressure being producible at the distal end of the catheter when the positive fluid pressure counterbalances that of the negative fluid pressure.

12. The method in accordance with claim 11, further comprising the step of maximizing at least one waveform parameter of the pulsatile aspiration waveform produced at the distal end of the catheter by controlling the pump system based on the resulting pressure monitored by a pressure sensor.

13. The method in accordance with claim 12, further comprising the step of:
if the resulting pressure monitored by the pressure sensor exceeds a predetermined threshold, setting, via the pump system, an adjusted level of the positive fluid pressure so that the resulting pressure monitored by the pressure sensor is reduced to or below the predetermined threshold.

14. The method in accordance with claim 11, further comprising the steps of:
capturing a targeted clot in a funnel section disposed on the distal end of the catheter, a distal end of the funnel section having a larger diameter relative to a proximal end of the funnel section;
detecting, via a second sensor disposed on an inner surface of the funnel section, a parameter of the targeted clot; and
based the detected parameter, adjusting at least one waveform parameter for controlling the pump system.

15. The method in accordance with claim 11, further comprising arranging the auxiliary lumen eccentrically of the central main lumen.

16. The method in accordance with claim 15, further comprising permanently fixing the internal wall in position within and dividing the proximal section of the passageway of the catheter into the central main lumen and the auxiliary lumen on opposite sides of the internal wall in a radial direction.

17. The method in accordance with claim 11, further comprising concentrically arranging the central main lumen and auxiliary lumen.

18. The method in accordance with claim 17, further comprising arranging the internal wall as an inner catheter within the proximal section of the passageway of the catheter and slidable in the longitudinal direction independently of the catheter; and
forming a channel in the inner catheter in the longitudinal direction defined therein serving as the central main lumen of the proximal section of the passageway of the catheter while a region defined in a radial direction between the inner catheter and the proximal section of the catheter serves as the auxiliary lumen.

19. The method in accordance with claim 12, wherein when the resulting pressure in the distal section of the passageway of the catheter monitored by the pressure sensor exceeds a predetermined threshold, further comprising the step of: (i) activating an indicator; and/or (ii) adjusting, via the pump system, at least one waveform parameter of the positive pressure fluid so that the monitored resulting pressure is below the predetermined threshold.

20. The method in accordance with claim 11, further comprising the step of introducing a stentriever via the central main lumen of the catheter.
